(19) United States
(12) Patent Application Publication
(10) Pub. No.: US 2018/0325677 A1
(43) Pub. Date: Nov. 15, 2018

(54) Annular ceramic insert for endoprosthetics

(71) Applicant: CeramTec GmbH, Plochingen (DE)

(72) Inventor: Norbert SCHNEIDER, Schorndorf (DE)

(21) Appl. No.: 15/776,100

(22) PCT Filed: Nov. 17, 2016
(86) PCT No.: PCT/EP2016/077941

§ 371 Date: May 15, 2018

(30) Foreign Application Priority Data
Nov. 19, 2015 (DE) 10 2015 222 818.5

(51) Int. Cl. A61F 2/34; F16C 11/06

(52) U.S. Cl. CPC A61F 2/34; A61F 2002/302; F16C 11/0623

(57) ABSTRACT

The invention relates to a ceramic insert (1) for sliding pairs in endoprosthetics. Said insert (1) has an outer face and an inner face; on the inner face, a hemispherical sliding area (2) is formed for accommodating a spherical sliding partner. In order to be able to reduce the thickness of the metal shell plus the insert such that the pelvic bone does not have to be milled as deep down, according to the invention, the insert (1) is designed as a ring or in an annular form.

The invention relates to a ceramic insert for sliding pairs in endoprosthetics, wherein the insert has an outer face and an inner face and a hemispherical sliding area is formed on the inner face for accommodating a spherical sliding partner.

Ceramic inserts for sliding pairs in hip endoprosthetics are embodied in hemispheres and cover approx. 50% of the prosthetic head. The center point of the sliding surface is disposed on the plane of the end face or slightly thereabove or therebelow.

Running on the equator of the outer face the insert has a more or less wide clamping surface (conical or cylindrical) with which it is inserted into a metal shell (preassembled or intraoperatively).

The rest of the back face to the pole is not in contact with the metal shell, but must have a minimum wall thickness for reasons of stability.

The load transmission between hip head and insert or acetabulum in the sliding surface is point shaped or arc shaped in this pair, since there is a positive clearance between the sphere diameter and cup diameter. The load is transmitted axis-parallel by the hip head onto the insert.

The underlying object of the invention is to improve a ceramic insert according to the preamble of claim 1 such that the width for metal shell plus insert is reduced so that the pelvic bone does not have to be milled as deep down. In addition, there should be no point-shaped loads, but rather there should be band-shaped loads with lower maximum values, similar to physiological loading. Furthermore, there should be a cost advantage due to savings in material and net volume during production (e.g., useable space in the furnace).

According to the invention, this object is attained using a ceramic insert according to the features of claim 1.

Since the insert is embodied as a ring or in an annular form, a component results that in its interior is a ring segment of a sphere. The posterior closure is preferably represented using radii that connect the inner face to the outer face. Because of this the insert has only approx. half the thickness of conventional inserts.

Advantages are:
Reduced width for shell plus insert, therefore the pelvic bone does not have to be milled as deep down.
No point-shaped loads, but instead band-shaped loads with lower maximum values, similar to physiological loading.
Savings in material and net volume during production (e.g. useable space in the furnace), resulting in cost advantage.

In one embodiment according to the invention, the inner face transitions via radii into the outer face. This applies both to the superior end and the inferior end of the insert. Thus there are no sharp edges, which improves stability.

The hemispherical sliding area preferably covers the entire inner face, so that the entire inner face is available for the sliding pairs. This reduces the width to a minimum.

In one advantageous embodiment, arranged on the outer face, at least in sub-areas, is a clamping surface that is embodied conically or cylindrically. This clamping surface provides anchoring in an outer metal shell.

This clamping surface on the outer face of the insert is preferably embodied conically or cylindrically, so that the insert may be securely connected to the metal shell.

The insert preferably has a width of 5-20 mm. At these widths little space is required and the clamping force is nevertheless unexpectedly adequate.

The inventive ceramic insert is embodied as a ring that on the top preferably has the same inner contour, end face, and clamping surface as conventional inserts, but only has a width of 5-20 mm.

Advantageous embodiments of the inventive ceramic insert are described in the figures.

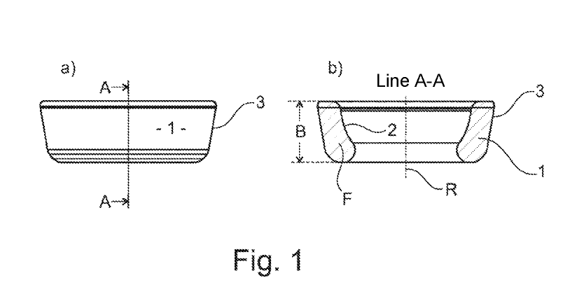

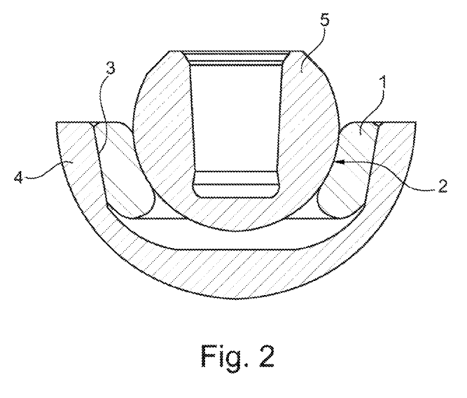

FIGS. 1a and 1b depict an inventive annular ceramic insert 1. FIG. 1a illustrates this insert 1 in one view and FIG. 1b illustrates it in section along the line A-A according to FIG. 1a. The insert 1 has an inner annular segment of a sphere, also called a hemispherical sliding area 2. In a hip prosthesis, the prosthetic head articulates thereon (see FIG. 2). Arranged on the outer face of the insert is a circumferential clamping surface 3 with which the insert may be anchored in a metal shell 4. The width of the insert B is between 5 and 20 mm. R identifies the axis of rotation. An annular embodiment results in an insert that deviates from the rotational symmetry in sub-areas.

FIG. 2 illustrates the insert 1 according to the invention inserted into a metal shell 4. A prosthesis head 5 has been inserted into the insert 1.

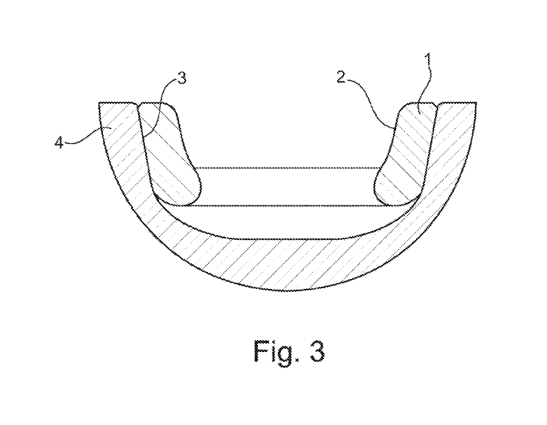

FIG. 3 is a sectional depiction of an insert 1 according to the invention inserted into a metal shell 4 and having an inner annular segment of a sphere or hemispherical sliding area 2. Reference number 3 identifies a preferably circumferential clamping surface. There may also be interruptions in the clamping surface.

A ring in this application shall be construed to be a body that is formed from one surface F (see FIG. 1b) that rotates around an axis of rotation R. The width B or the ring shall be construed to mean its extension along the axis of rotation R. Annular shall be construed to mean a ring that is embodied deviating from rotational symmetry in sub-areas.

1. A ceramic insert for sliding pairs in endoprosthetics, wherein the insert has an outer face and an inner face, and a hemispherical sliding area is formed on the inner face for accommodating a spherical sliding partner, wherein the insert is designed as a ring or in an annular form.

2. The insert according to claim 1, wherein insert has a width (B) of 5 to 20 mm.

3. The insert according to claim 1, wherein the inner face transitions via radii into the outer face.

4. The insert according to claim 1, wherein the hemispherical sliding area covers the entire inner face.

5. The insert according to claim 1, wherein arranged on the outer face, at least in sub-areas, is a clamping surface that is embodied conically or cylindrically.

6. An application of an insert according to claim 1 in hip, shoulder, toe, or knee endoprosthetics.

7. A hip endoprosthetic comprising the ceramic insert according to claim 1.

8. A shoulder endoprosthetic comprising the ceramic insert according to claim 1.

9. A toe endoprosthetic comprising the ceramic insert according to claim 1.

10. A knee endoprosthetic comprising the ceramic insert according to claim 1.
